(19) United States
(12) Patent Application Publication
(10) Pub. No.: US 2025/0131852 A1
(43) Pub. Date: Apr. 24, 2025

(54) ASSEMBLIES AND METHODS FOR PERFORMING PRACTICE EXERCISES WITH A SURGICAL DEVICE

(71) Applicant: MOMENTIS SURGICAL LTD., Or-Yehuda (IL)

(72) Inventors: Yaron LEVINSON, Tel Aviv (IL); Efrat MILMAN, Tel Aviv (IL)

(21) Appl. No.: 18/571,989

(22) PCT Filed: Jun. 27, 2022
(86) PCT No.: PCT/IB2022/055919

§ 371 Date: Dec. 19, 2023

Related U.S. Application Data
(60) Provisional application No. 63/215,586, filed on Jun. 28, 2021.

(51) Int. Cl. G09B 23/28

(52) U.S. Cl. CPC G09B 23/285

(57) ABSTRACT

The present invention relates to assemblies and methods for performing practice exercises with a surgical device, and in particular with a surgical device comprising one or more mechanical arms that are articulated and/or flexible. Some embodiments relate to an assembly for performing practice exercises with a surgical device that comprises one or more articulated arms comprising: a. a support platform defining a platform plane; b. an exercise-element carrier raised above the platform plane and comprising a first practice surface, the first practice surface comprising an array of one or more practice-exercise elements selected from the group consisting of holes, loops, pegs and hooks; and c. an aperture raised above the platform plane and framed at least along an upper edge of the aperture, the aperture shaped for passage therethrough of a distal portion of an articulated arm.

### FIELD OF THE INVENTION

The present invention relates to assemblies and methods for performing practice exercises with a surgical device, and in particular with a surgical device comprising one or more mechanical arms that are articulated and/or flexible.

### BACKGROUND

It is well established that there are benefits of minimally invasive surgery. Instruments for such surgery typically have a surgical end effector located at the distal end of an articulated surgical arm (preferably with minimum diameter) that is inserted through a small opening (e.g., body wall incision, natural orifice) to reach a surgical site. In some instances, surgical instruments can be passed through a cannula and an endoscope can be used to provide images of the surgical site.

Surgical instruments have been developed that utilize an end effector (e.g., a surgical tool such as for tissue fusing or cutting, or a measurement tool) for convenience, accuracy, and wellbeing of the subject. In some cases, articulated surgical arms have one or more bending portions which are controlled remotely using various input devices (e.g., hand and foot controls) to ultimately control the location of the end effector and change its orientation with reference to the surgical arm's longitudinal axis. In some case, the surgical arm is capable of retroflected bending relative to the surgical arm longitudinal axis.

The current state of the art is lacking devices and methods that can provide opportunities for performing practice exercises with physical exercise elements for purposes of demonstration of the surgical apparatus and/or training the use of the surgical apparatus. Simulators and generic collections of exercise elements may fail to provide a proper physical environment suitable for performing practice exercises with specific surgical devices having respective ergonomics. Thus, there is a need for suitable solutions for performing practice exercises with specific surgical devices, such as those with articulated and/or flexible mechanical arms.

### SUMMARY OF THE INVENTION

According to embodiments disclosed herein, an assembly for performing practice exercises with a surgical device that comprises one or more articulated arms comprises: (a) a support platform defining a platform plane; (b) an exercise-element carrier raised above the platform plane and comprising a first practice surface, the first practice surface comprising an array of one or more practice-exercise elements selected from the group consisting of holes, loops, pegs and hooks; and (c) an aperture raised above the platform plane and framed at least along an upper edge of the aperture, the aperture shaped for passage therethrough of a distal portion of an articulated arm. The exercise-element carrier and the aperture are arranged so that a practice-exercise element of the first practice surface is accessible to a distal end effector of the articulated arm when the articulated arm is in a retroflex position.

In some embodiments, it can be that the practice-exercise element of the first practice surface is accessible by the distal end effector of the articulated arm only when the articulated arm is in in the retroflex position.

In some embodiments, the first practice surface can be oriented at an angle between 45° and 135° relative to the platform plane.

In some embodiments, the retroflex position can be such that the articulated arm is flexed by at least 120° from an unflexed orientation. In some embodiments, the retroflex position can be such that the articulated arm is flexed by at least 150° from an unflexed orientation. In some embodiments, the retroflex position can be such that the articulated arm is flexed by at least 180° from an unflexed orientation.

In some embodiments, the aperture can be formed through, under, and/or atop, the exercise-element carrier. In some embodiments, the upper edge of the aperture can be below the array of one or more practice-exercise elements of the first practice surface. In some embodiments, a framed lower edge of the aperture can be above the array of one or more practice-exercise elements of the first practice surface.

In some embodiments, it can be that the exercise-element carrier additionally comprises a second practice surface, wherein the second practice surface comprises an array of one or more practice-exercise elements selected from the group consisting of holes, loops, pegs and hooks, and/or wherein the array of one or more practice-exercise elements of the second practice surface is accessible to the distal end effector of the articulated arm when the articulated arm is not in a retroflex position.

In some embodiments, it can be that the first and second practice surfaces are parallel. In some embodiments, it can be that the first and second practice surfaces are oriented at an acute angle to each other.

In some embodiments, the second practice surface can be oriented at an angle between 45° and 135° relative to the platform plane.

In some embodiments, a practice-exercise element of the second practice surface can be accessible to the distal end effector of the articulated arm when the articulated arm is in an S-shaped position.

In some embodiments, a method for performing practice exercises with the surgical device can comprise: (a) providing an assembly according to any of the embodiments disclosed hereinabove wherein the exercise-element carrier comprises a first practice surface; (b) causing the distal portion of the articulated arm to traverse the aperture; and (c) with the articulated arm in the retroflex position, causing the distal end effector of the articulated arm to perform a practice exercise with at least one practice-exercise element of the first practice surface.

In some embodiments, the method can additionally comprise, before the causing of the distal end effector of the articulated arm to perform the practice exercise: orienting the exercise-element carrier so that the first practice surface is set to an angle between 45° and 135° relative to the platform plane.

In some embodiments, the method can additionally comprise, after the causing of the distal portion of the articulated arm to traverse the aperture, and before the causing of the distal end effector of the articulated arm to perform the practice exercise: retroflexing the articulated arm.

In some embodiments, a method of performing practice exercises with a surgical device that comprises one or more articulated arms can comprise: (a) providing an assembly according to any of the embodiments disclosed hereinabove wherein the exercise-element carrier additionally comprises a second practice surface; (b) at a first time: (i) causing the distal portion of the articulated arm to traverse the aperture, and (ii) with the articulated arm in the retroflex position, causing the distal end effector of the articulated arm to perform a practice exercise with at least one of the practice-exercise elements of the first practice surface; and (c) at a second time, with the articulated arm not in a retroflex position, causing the distal end effector of the articulated arm to perform a practice exercise with at least one of the practice-exercise elements of the second practice surface.

In some embodiments, the method can additionally comprise, before the causing of the distal end effector of the articulated arm to perform the practice exercise with the at least one of the practice-exercise elements of the second practice surface: orienting the exercise-element carrier so that the second practice surface is set to an angle between 45° and 135° relative to the platform plane.

According to embodiments disclosed herein, an assembly for performing practice exercises with a surgical device that comprises one or more articulated arms comprises: (a) a support platform defining a platform plane; (b) a proximal exercise-element carrier raised above the platform plane and comprising a distally-facing practice surface, the proximal exercise-element carrier having formed therethrough or thereunder an aperture shaped for passage therethrough of a distal portion of an articulated arm; and (c) a distal exercise-element carrier raised above the platform plane and comprising a proximally-facing practice surface. The aperture and the proximal and distal exercise panels are arranged so that (i) a distally-disposed practice-exercise element of the proximal exercise panel, selected from the group consisting of holes, loops, pegs and hooks, is accessible to a distal end effector of the articulated arm when the articulated arm is in a retroflex position, and (ii) a proximally-disposed practice-exercise element of the distal exercise panel, selected from the group consisting of holes, loops, pegs and hooks, is accessible to the distal end effector of the articulated arm when the articulated arm is not in a retroflex position.

In some embodiments, it can be that the distally-disposed practice-exercise element of the proximal exercise-element carrier is accessible to the distal end effector of the articulated arm only when the articulated arm is in a retroflex position.

In some embodiments, the respective practice surfaces of the proximal and distal exercise panels can be oriented at respective angles between 45° and 135° relative to the platform plane.

In some embodiments, the retroflex position can be such that the articulated arm is flexed by at least 120° from an unflexed orientation. In some embodiments, the retroflex position can be such that the articulated arm is flexed by at least 150° from an unflexed orientation. In some embodiments, the retroflex position can be such that the articulated arm is flexed by at least 180° from an unflexed orientation.

In some embodiments, an upper edge of the aperture can be below the at least one practice-exercise element of the distally-facing practice surface of the proximal exercise panel. In some embodiments, a lower edge of the aperture can be above the at least one practice-exercise element of the proximally-facing practice surface of the distal exercise panel.

In some embodiments, the proximally-disposed practice-exercise element of the distal exercise-element carrier can be accessible to the distal end effector of the articulated arm when the articulated arm is in an S-shaped position.

In some embodiments, a method of performing practice exercises with the surgical device comprising one or more articulated arms comprises: (a) providing an assembly according to any of the embodiments disclosed hereinabove in which the assembly comprises respective proximal and distal exercise panels; (b) at a first time: (i) causing the distal portion of the articulated arm to traverse the aperture, and (ii) with the articulated arm in the retroflex position, causing the distal end effector of the articulated arm to perform a practice exercise with a distally-disposed practice-exercise element of the proximal exercise panel; and (c) at a second time, with the articulated arm not in a retroflex position, causing the distal end effector of the articulated arm to perform a practice exercise with a proximally-disposed practice-exercise element of the distal exercise panel.

In some embodiments, the method can additionally comprise, before the causing of the distal end effector of the articulated arm to perform the practice exercise with the distally-disposed practice-exercise element of the proximal exercise panel: orienting the proximal exercise-element carrier so that the distally-facing practice surface is set to an angle between 45° and 135° relative to the platform plane.

In some embodiments, the method can additionally comprise, before the causing of the distal end effector of the articulated arm to perform the practice exercise with the proximally-disposed practice-exercise element of the distal exercise panel: orienting the distal exercise-element carrier so that the proximally-facing practice surface is set to an angle between 45° and 135° relative to the platform plane.

In some embodiments, the method can additionally comprise, at the first time, after the causing of the distal portion of the articulated arm to traverse the aperture and before the causing of the distal end effector of the articulated arm to perform the practice exercise with the distally-disposed practice-exercise element of the proximal exercise panel: retroflexing the articulated arm.

### BRIEF DESCRIPTION OF THE DRAWINGS

The invention will now be described further, by way of example, with reference to the accompanying drawings, in which the dimensions of components and features shown in the figures are chosen for convenience and clarity of presentation and not necessarily to scale. In the drawings:

FIG. 1 is a schematic perspective view of a surgical device comprising two mechanical arms.

FIGS. 2A, 2B and 2C are schematic views of a support platform defining platform plane, according to embodiments of the present invention.

FIGS. 3A and 3B are schematic views of exercise-element carriers, according to embodiments of the present invention.

FIG. 4 shows a variety of practice-exercise elements, according to embodiments of the present invention.

FIG. 5 is a schematic side view of an assembly comprising a support platform and an exercise-element carrier pivotable through a pivot range, according to embodiments of the present invention.

FIG. 6 is a schematic diagram showing a mechanical arm and a variety of retroflex angles.

FIGS. 7A, 7B, 7C and 7D are schematic end views of examples of assemblies comprising respective support platforms, exercise-element carriers and apertures, according to embodiments of the present invention.

FIGS. 8A and 8B are schematic side views of a surgical device comprising a mechanical arm having an end effector accessing, in retroflex position, a practice-exercise element of an exercise-element carrier, according to embodiments of the present invention.

FIGS. 9A and 9B are schematic side views of a surgical device comprising a mechanical arm having an end effector accessing, not in retroflex position, a practice-exercise element of an exercise-element carrier, according to embodiments of the present invention.

FIGS. 10A and 10B are schematic side views, shown respectively at a first time and at a second time, of a surgical device comprising a mechanical arm having an end effector accessing, at the first time and in retroflex position, a practice-exercise element of a first practice surface of an exercise-element carrier having two opposing practice surfaces, and at the second time and not in the retroflex position, a practice-exercise element of the second practice surface, according to embodiments of the present invention.

FIGS. 11A and 11B are schematic side views, shown respectively at a first time and at a second time, of a surgical device comprising a mechanical arm having an end effector accessing, at the first time and in retroflex position, a practice-exercise element of a first practice surface of an exercise-element carrier having two practice surfaces set apart from each other at an acute angle, and at the second time and not in the retroflex position, a practice-exercise element of the second practice surface, according to embodiments of the present invention.

FIGS. 12A and 12B are schematic side views, shown respectively at a first time and at a second time, of a surgical device comprising a mechanical arm having an end effector accessing, at the first time and in retroflex position, a practice-exercise element of a first exercise-element carrier, and at the second time and not in the retroflex position, a practice-exercise element of a second exercise-element carrier, according to embodiments of the present invention.

FIG. 13A is a schematic side view of an assembly comprising a support platform, an exercise-element carrier, and an aperture that is not parallel to the exercise-element carrier, according to embodiments of the present invention.

FIG. 13B is a schematic perspective view of a surgical device comprising a mechanical arm having an end effector accessing, through the aperture of FIG. 13A and not in the retroflex position, a practice-exercise element of the exercise-element carrier of FIG. 13A, according to embodiments of the present invention.

FIG. 14 a schematic diagram showing a flexed mechanical arm.

FIGS. 15A, 15B, 15C, 16A, 16B, 17A, 17B, 17C and 17D show flowcharts of methods and method steps for performing practice exercises with a surgical device that comprises one or more articulated arms, according to embodiments of the present invention.

### DETAILED DESCRIPTION OF EMBODIMENTS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are generally used to designate like elements.

Note: Throughout this disclosure, subscripted reference numbers (e.g., 10₁ or 10_{A}) may be used to designate multiple separate appearances of elements of a single species, whether in a drawing or not; for example: 10₁ is a single appearance (out of a plurality of appearances) of element 10. The same elements can alternatively be referred to without subscript (e.g., 10 and not 10₁) when not referring to a specific one of the multiple separate appearances, i.e., to the species in general.

Embodiments disclosed herein relate to controlling one or more surgical mechanical arms, i.e., articulated mechanical arms, using a plurality of different operating modes and/or a plurality of different input devices.

Whenever ‘arm’ is used herein or in the appended claims, it means an articulated, mechanical arm that is part of a surgical system or electrosurgical system and used for performing or helping to perform surgical (including electrosurgical) actions inside a human subject's body. An articulated arm can also be considered flexible. An arm may include an end effector, which is used herein to mean a tool or device used in connection with surgery, electrosurgery, diagnosis or imaging when deployed within a human body. An end effector may be supplied as part of an arm, i.e., already mounted, mechanical attached and/or integrated with the power and communications conveyances of the arm; in some embodiments an arm an end effector may be provided separately for assembly and/or integration into a working unit before or even during a surgical operation, i.e., before insertion into a subject's body. In any case, terms such as ‘an arm comprising an end effector’ and ‘and arm configured for use with an end effector’, etc., should be understood as equivalent for the purposes of this disclosure and the claims appended thereto. A ‘surgical device’ as used herein means a device having one or more surgical mechanical arms and a motor unit or motor-control unit for controlling the one or more arms.

The term ‘distal’ as used herein and in the claims appended herewith means further from a motor-control unit of a surgical device, and the term ‘proximal’ means closer to the motor-control unit of the surgical device.

‘Practice exercises’ as used herein, means use of surgical devices, and particularly of one or more surgical mechanical arms of surgical devices, in non-medical exercises for purposes such as, without limitation, demonstration or training the use of the surgical device and arm(s).

An assembly for performing practice exercises using the surgical device, i.e., using the one or more surgical arms, according to embodiments of the present invention, can include one or more exercise-element carrier each having one either one or two practice surfaces raised above a support platform. The practice surfaces can comprise and/or have attached thereto any number of practice-exercise elements accessible in various configurations by an end-effector of a surgical arm. Some practice elements used in connection with the exercise-element carriers are integral, i.e., formed in or permanently attached to, an exercise-element carrier. An example of integral practice elements include holes formed in an exercise-element carrier for the purpose of inserting pegs into the holes. The holes would be an example of attachable/detachable practice-exercise elements. In some examples, attachable/detachable practice-exercise elements are only either attachable or detachable by an end effector of a surgical arm, and the practice-exercise action can be either permanently attached or detached, or the attaching or detaching is reversible only by operator intervention.

‘Accessible’ as used herein means that the surgical arm can be positioned, extended, retracted, flexed and/or retroflexed so as to put a respective end-effector in contact with a practice-exercise element and perform a practice exercise with it. The performing of a practice exercise can include any one or more of, and not exhaustively: moving, turning, rotating, pulling, pushing, removing (detaching) from a practice surface, inserting (attaching) to a practice surface, hooking, unhooking, looping, unlooping one or more practice-exercise elements.

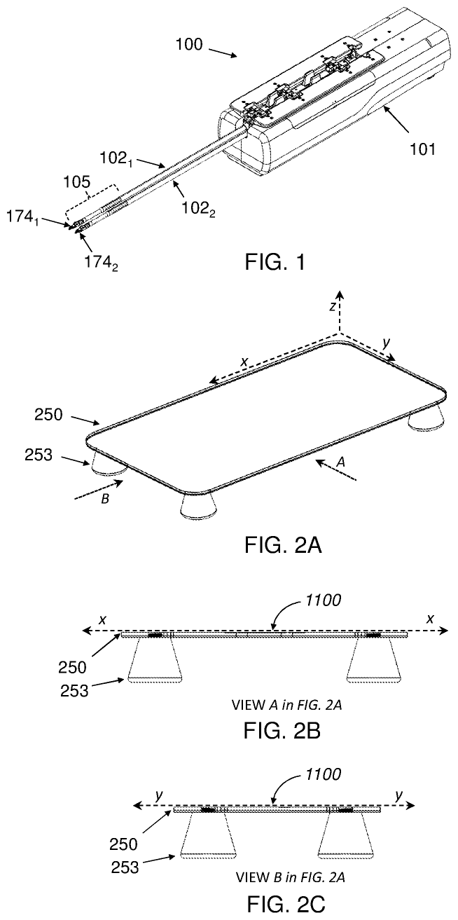

Referring now to the figures and in particular to FIG. 1, non-limiting example of a surgical device 100 is shown. The surgical device 100 includes a motor-control unit 101, and two arms 102₁, 102₂. Each arm 102 has a respective end effector 174 distally installed at the tip of a respective distal portion 105. In embodiments, a motor-control unit 101 is effective to control the movement of an arm 102 by extending, retracting, and/or flexing the arm 102 so as to move the end effector 174 through three-dimensional space.

FIGS. 2A, 2B and 2C illustrate a non-limiting example of a support platform 250 suitable for performing practice exercises thereupon. The support platform 250 can stand, as illustrated, on legs 253 that facilitate the securing of elements to the support platform 250, including, for example, securing from below. The support platform defines a platform plane 1100 in the x-y plane according to the x-y-z axes shown in FIG. 2A. In the illustrated example, the x-y plane is horizontal, i.e., parallel to a floor, but in other examples, the x-y plane and therefore the platform plane 1100 defined by the support platform 250 can be at an angle to a floor—for example if some legs 253 are longer than other, or if the platform 250 is placed on a non-level or uneven surface.

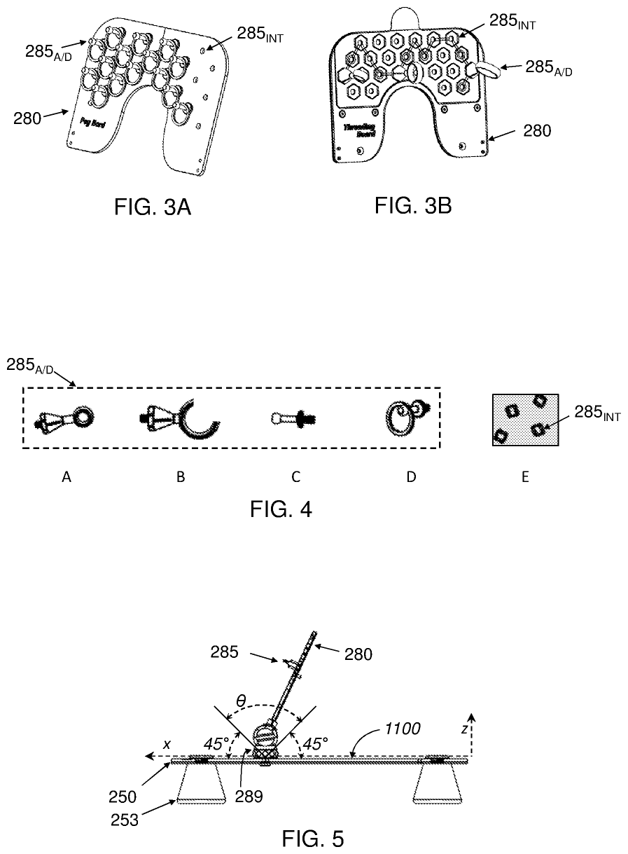

FIGS. 3A and 3B show non-limiting examples of exercise-element carriers 280. The exercise-element carrier 280 of FIG. 3A includes an array of integral practice-exercise elements 285_{INT} in the form of small holes, and an array of attachable/detachable practice-exercise elements 285_{A/D} in the form of pin-and-loop assemblies inserted into additional small holes 285_{INT}. The exercise-element carrier 280 of FIG. 3B includes a different type of integral exercise elements 285_{INT} in the form of small holes formed within hexagonal recesses, such that it can be seen that the practice exercise of inserting a peg into such a hole would involve not only attaching but also rotating so as to line up with the hexagonal recess. Accordingly, the attachable/detachable practice-exercise elements 285_{A/D} shown attached to the exercise-element carrier 280 of FIG. 3B have hexagonal bases.

FIG. 4 shows a number of non-limiting examples of practice-exercise elements 285 including attachable/detachable practice-exercise elements 285_{A/D} (examples A, B, C and D) and integral practice-exercise elements 285_{INT} (in example E). Practice-exercise elements 285 can be of any size or shape suitable for performing therewith practice exercises performable by any specific surgical end-effector 174 of an articulated surgical arm 102.

FIG. 5 is a side view of an assembly comprising a support platform 250 and a exercise-element carrier 280 raised above the support platform 250. ‘Raised above’ as used in the disclosure and in the claims appended thereto means raised in the z-axis direction above the platform plane 1100 which is in the x-y plane. FIG. 5 illustrates an exemplary arrangement in which the exercise-element carrier 280 (having attached thereto a practice-exercise element 285) is affixed to the support platform 250 by pivotable base element 289 which allows the support platform 250 to pivot within a range of ±θ (e.g., in degrees) from the z-axis vertical direction. Specifically, it can be desirable to provide a pivotable base element 289 which allows the support platform 250 to pivot within a range of +45° from the z-axis vertical direction, i.e., between 45° and 135°, for optimal positioning relative to a surgical arm 102 of surgical device 100.

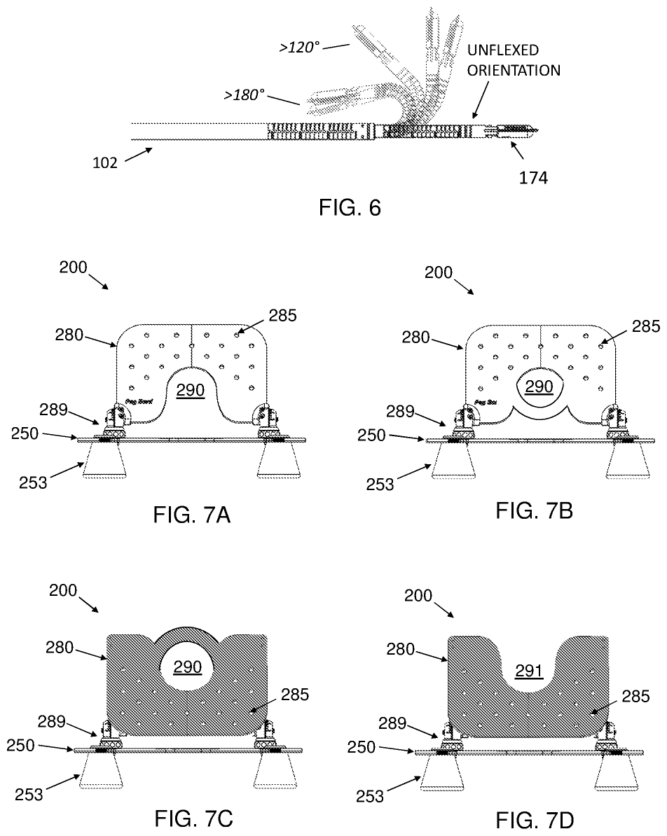

FIG. 6 schematically illustrates the flexing ability of an articulated arm 102 according to embodiments. The arm 102 and its end effector 174 can be placed in a retroflex position which includes being flexed at least 120° from an unflexed position, or at least 150° from an unflexed position, or at least 180° from an unflexed position. The flexing can be performed by a single joint of the articulated arm 102 or by more than one joint operating in combination.

Some practice exercises include causing the end effector 174, i.e., the distal end or a distal portion 105 of a surgical mechanical arm 102 to pass through an aperture 290 before accessing a practice-exercise element 285 on an exercise-element carrier 280 that comprises an array of practice-exercise elements 285. A number of non-limiting examples of aperture configurations are illustrated in FIGS. 7A, 7B, 7C, and 7D.

FIG. 7A illustrates a type of aperture 290 formed in the exercise-element carrier 280, in which an upper edge of the aperture 290 is below at least one practice-exercise element 285 of the array of practice-exercise elements 285. The aperture 290 of FIG. 7A is bounded on a lower edge only by the support platform 250. The aperture 290 of FIG. 7B is similarly formed in the exercise-element carrier 280 and similarly positioned to have an upper edge of the aperture 290 be below at least one practice-exercise element 285 of the array of practice-exercise elements 285, but the aperture 290 of FIG. 7B is bounded on a lower edge by part of the surface of the exercise-element carrier 280.

FIG. 7C illustrates another type of aperture 290 formed in the exercise-element carrier 280, in which a lower edge of the aperture 290 is disposed above at least one practice-exercise element 285 of the array of practice-exercise elements 285. The aperture 290 of FIG. 7A is bounded on an upper edge by a part of the surface of the exercise-element carrier 280. The aperture 290 of FIG. 7D is similarly formed in the exercise-element carrier 280 and similarly positioned to have a lower edge of the aperture 290 be above at least one practice-exercise element 285 of the array of practice-exercise elements 285, but the aperture 290 of FIG. 7B is not bounded on its upper edge.

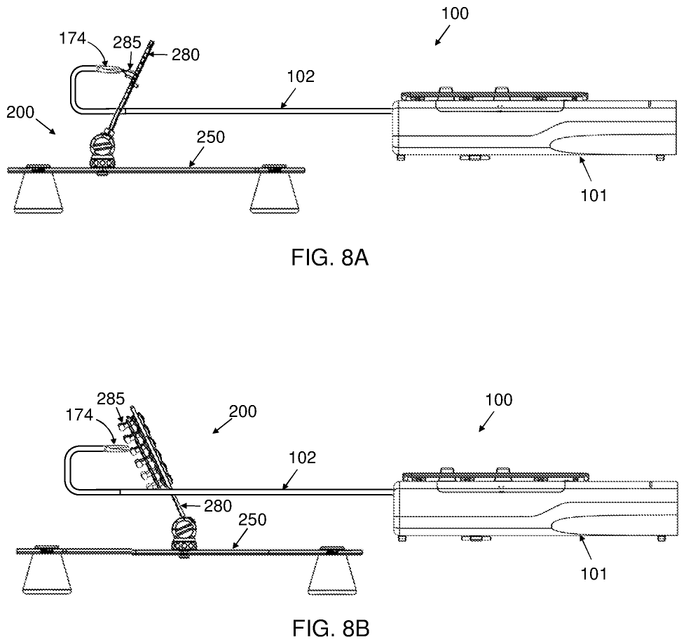

FIGS. 8A and 8B are schematic illustrations of a surgical device 100 positioned proximally from a practice-exercise assembly 200 to access a practice-exercise element 285 arranged on exercise-element carrier 280. The angle at which the exercise-element carrier 280 stands relative to the support platform 250 differs between FIGS. 8A and 8B to illustrate two types of practice exercises that may be performed in this configuration. Note: In any of the figures appended herewith, the relative sizes of the surgical device 100, including its components such as the motor-control unit 101 and the surgical arm(s) 102, and of the practice-exercise assembly 200 and its components are not drawing to scale. Moreover, the positioning of the surgical device 100 is not intended to represent a necessary or even typical positioning. In both FIGS. 8A and 8B, the arm 102 is in a retroflex position of at least 180° relative to an unflexed position. It can be understood from the two figures that, in these examples, the practice-exercise element 285 is accessible to the end effector 174 only when the arm 102 is in the retroflex position, as there is no direct access to the practice-exercise element 285 without going over, under, through, or around the exercise-element carrier 280.

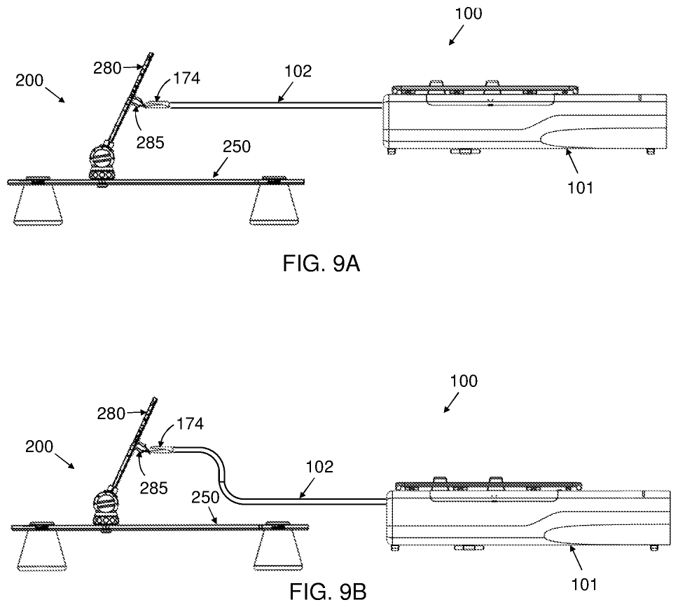

FIGS. 9A and 9B illustrate examples of accessing, with an end effector 174, a ‘directly-accessible’ practice-exercise element 285, i.e., a practice-exercise element 285 that is accessible without the arm 102 being in a retroflex position. In the example of FIG. 9A, the arm is not flexed at all to afford the end effector 174 direct access to the practice-exercise element 285, while in the example of FIG. 9B, the arm 102 is flexed into an ‘S’-shaped position so to access the practice-exercise element 285. The difference in arm shape 102 may be a factor of distance of the surgical device 100 from the practice-exercise assembly 200, or it may be, for example, in response to an instruction to train the use of specifically flexing the arm 102 into a complex position before performing the practice exercise illustrated.

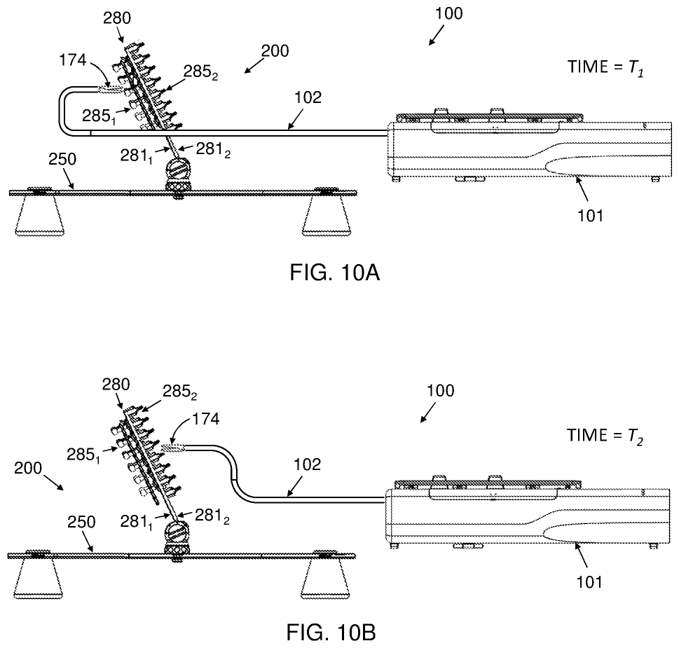

FIGS. 10A and 10B are schematic side views, shown respectively at a first time (Time=T₁) and at a second time (Time=T₂), of a surgical device 100 comprising a mechanical arm 102 having an end effector 174 accessing, at Time=T₁ and in retroflex position, a practice-exercise element 285₁ of a first practice surface 281₁ of an exercise-element carrier 280 having two opposing practice surfaces 281₁, 281₂, and at Time=T₂ and not in the retroflex position, a practice-exercise element 285₂ of the second practice surface 281₂. In some embodiments, the surgical device 100 comprises one arm 102 and the same arm 102 and same end effector 174 are used for both of the practice exercises illustrated by FIGS. 10A and 10B. In some embodiments, the surgical device 100 comprises multiple arms 102, and the practice exercises illustrated by FIGS. 10A and 10B are performed by different arms 102. In some such embodiments employing multiple arms 102, the times T₁ and T₂ can be the same time.

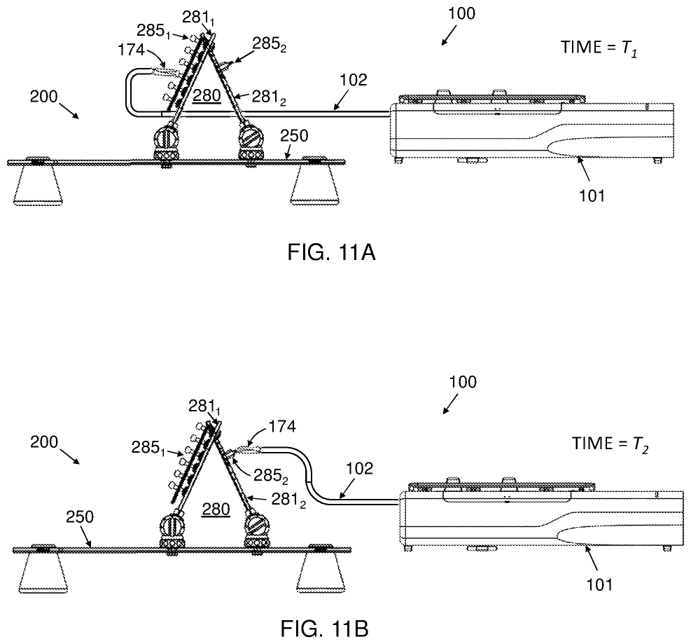

FIGS. 11A and 11B are schematic side views, shown respectively at a first time (Time=T₁) and at a second time (Time=T₂), of a surgical device 100 comprising a mechanical arm 102 having an end effector 174 accessing, at Time=T₁ and in retroflex position, a practice-exercise element 285₁ of a first practice surface 281₁ of an exercise-element carrier 280 having two practice surfaces 281₁, 281₂ disposed at an acute angle from each other, and at Time=T₂ and not in the retroflex position, a practice-exercise element 285₂ of the second practice surface 281₂. In some embodiments, the surgical device 100 comprises one arm 102 and the same arm 102 and same end effector 174 are used for both of the practice exercises illustrated by FIGS. 11A and 11B. In some embodiments, the surgical device 100 comprises multiple arms 102, and the practice exercises illustrated by FIGS. 11A and 11B are performed by different arms 102. In some such embodiments employing multiple arms 102, the times T₁ and T₂ can be the same time.

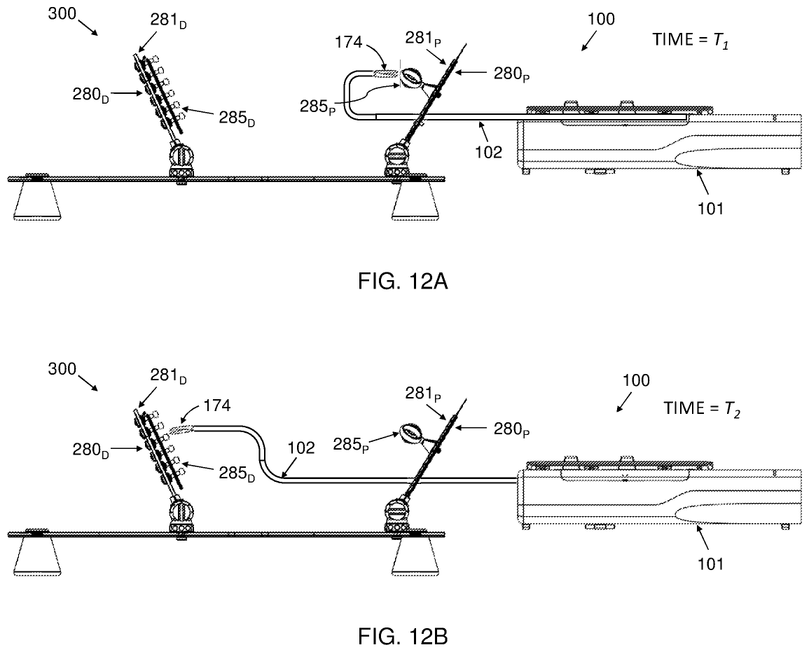

In some embodiments, the practice-exercise assembly 200 can include multiple exercise-element carriers 280—in the non-limiting example of FIGS. 12A and 12B, a proximally-disposed exercise-element carrier 280_{P} and a distally-disposed exercise-element carrier 280_{D}. In FIGS. 12A, the proximally-disposed exercise-element carrier 280_{P} is shown as having a distally-facing practice surface 281_{P}, while the distally-disposed exercise-element carrier 280_{D} is shown as having a proximally-facing practice surface 281_{D}. (For clarity: the subscript of the practice surface 281 in FIGS. 12A and 12B) matches that of the exercise-element carrier 280.) In other examples, of course, a proximally-disposed exercise-element carrier 280_{P} can have a proximally-facing practice surface 281_{P}, while the distally-disposed exercise-element carrier 280_{D} can have a distally-facing practice surface 281_{D}.

FIGS. 12A and 12B are schematic side views, shown respectively at a first time (Time=T₁) and at a second time (Time=T₂), of a surgical device 100 comprising a mechanical arm 102 having an end effector 174 accessing, at Time=T₁ and in retroflex position, a practice-exercise element 285_{P} of the proximally-disposed exercise-element carrier 280_{P}, and at Time=T₂ and not in the retroflex position, a practice-exercise element 285_{D} of distally-disposed exercise-element carrier 280_{D}. In some embodiments, the surgical device 100 comprises one arm 102 and the same arm 102 and same end effector 174 are used for both of the practice exercises illustrated by FIGS. 12A and 12B. In some embodiments, the surgical device 100 comprises multiple arms 102, and the practice exercises illustrated by FIGS. 12A and 12B are performed by different arms 102. In some such embodiments employing multiple arms 102, the times T₁ and T₂ can be the same time.

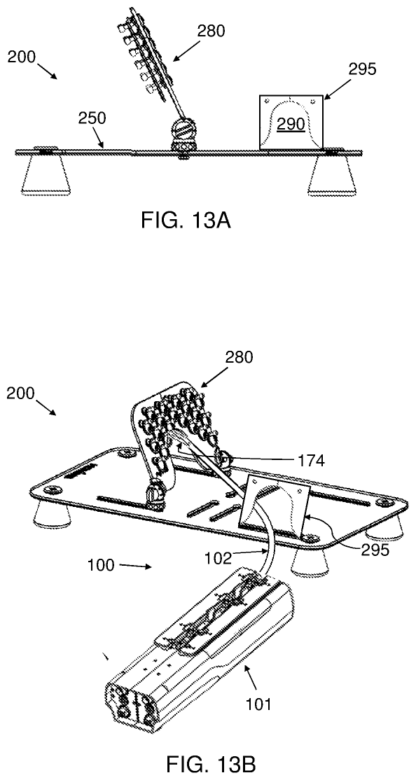

We now refer to FIGS. 13A and 13B. In some embodiments, an aperture is formed not under, over or through exercise-element carrier 280. FIG. 13A is a schematic side view of a practice-exercise assembly 200 comprising a support platform 250, an exercise-element carrier 280, and an aperture 290 that is neither formed in nor parallel to the exercise-element carrier 280. FIG. 13B shows the assembly 200 of FIG. 13A with a surgical device 100 comprising a mechanical arm 102 having an end effector 174 accessing, through the aperture 290 and not in the retroflex position, a practice-exercise element 285 of the exercise-element carrier 280.

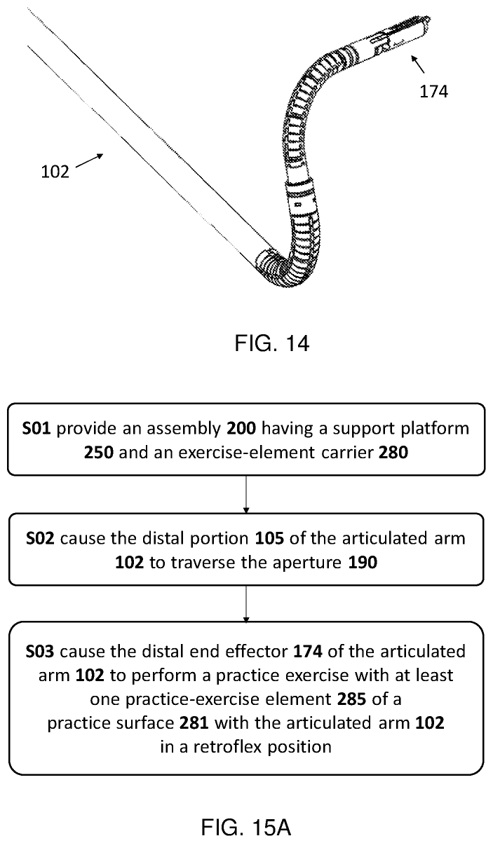

FIG. 14 a schematic diagram showing a flexed mechanical arm 102 in a non-limiting example of a non-retroflex, complex orientation/position of an arm 102 that may be used in perform practice exercises in connection with any of the examples disclosed herein which do not require the arm 102 to be in the retroflex position.

Methods are now disclosed of performing practices exercises with any of the practice-exercise assemblies 200 disclosed herein.

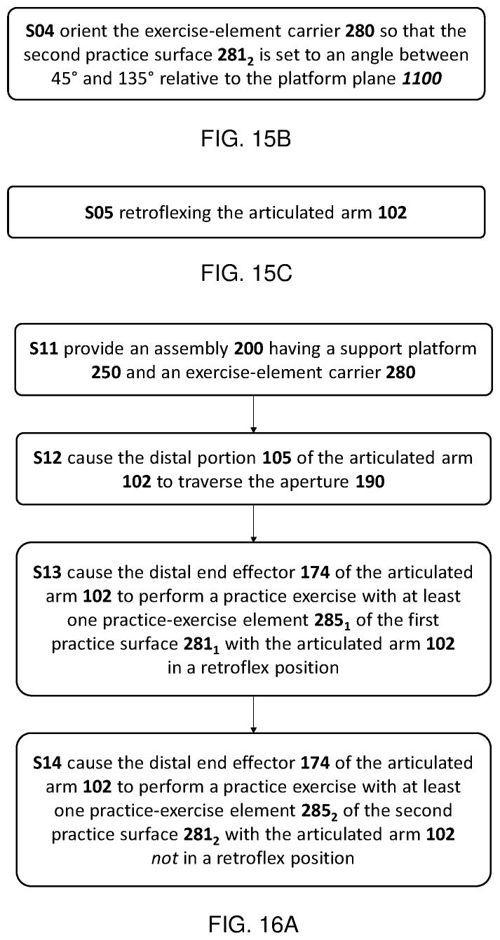

In embodiments, as shown in the flowchart of FIG. 15A, a method for performing practice exercises with the surgical device 100 comprises:

Step S01 provide an assembly 200 (according to any of the embodiments disclosed hereinabove wherein the exercise-element carrier 280 comprises a first practice surface 281) having a support platform 250 and an exercise-element carrier 280

Step S02 cause distal portion 105 of the articulated arm 102 to traverse the aperture 190; and

Step S03 cause the distal end effector 174 of the articulated arm 102 to perform a practice exercise with at least one practice-exercise element 285 of a practice surface 281 with the articulated arm 102 in a retroflex position.

In some embodiments, as shown in the flowchart of FIG. 15B, the method can additionally comprise, before Step S03:

Step S04 orient the exercise-element carrier 280 so that the second practice surface 281₂ is set to an angle between 45° and 135° relative to the platform plane 1100.

In some embodiments, as shown in the flowchart of FIG. 15C, the method can additionally comprise, after Step S02 and before Step S03:

Step S05 retroflexing the articulated arm 102.

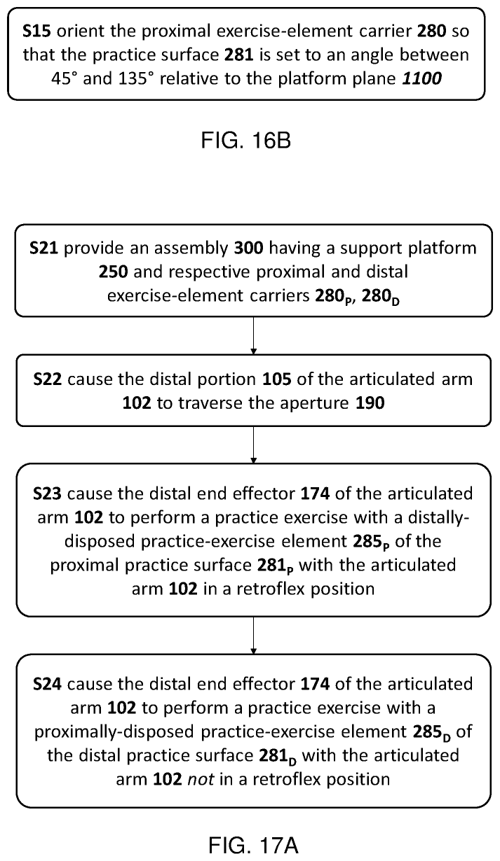

In embodiments, as shown in the flowchart of FIG. 16A, a method for performing practice exercises with the surgical device 100 comprises:

Step S11 provide an assembly 200 (according to any of the embodiments disclosed hereinabove wherein the exercise-element carrier 280 comprises first and practice surfaces 281₁, 281₂) having a support platform 250 and an exercise-element carrier 280;

Step S12 cause the distal portion 105 of the articulated arm 102 to traverse the aperture 190

Step S13 cause the distal end effector 174 of the articulated arm 102 to perform a practice exercise with at least one practice-exercise element 285₁ of the first practice surface 281₁ with the articulated arm 102 in a retroflex position; and

Step S14 cause the distal end effector 174 of the articulated arm 102 to perform a practice exercise with at least one practice-exercise element 285₂ of the second practice surface 281₂ with the articulated arm 102 not in a retroflex position

In some embodiments, as shown in the flowchart of FIG. 16B, the method can additionally comprise, before Step S13:

Step S15 orient the proximal exercise-element carrier 280 so that the practice surface 281 is set to an angle between 45° and 135° relative to the platform plane 1100.

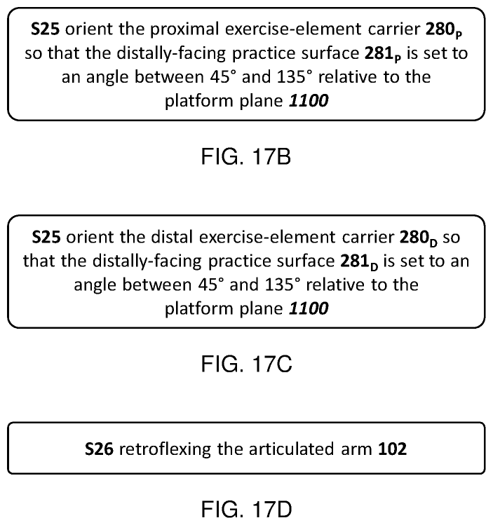

In embodiments, as shown in the flowchart of FIG. 17A, a method for performing practice exercises with the surgical device 100 comprises:

Step S21 provide an assembly 300 having a support platform 250 and respective proximal and distal exercise-element carriers 280_{P}, 280_{D};

Step S22 cause the distal portion 105 of the articulated arm 102 to traverse the aperture 190;

Step S23 cause the distal end effector 174 of the articulated arm 102 to perform a practice exercise with a distally-disposed practice-exercise element 285_{P} of the proximal practice surface 281_{P} with the articulated arm 102 in a retroflex position; and

Step S24 cause the distal end effector 174 of the articulated arm 102 to perform a practice exercise with a proximally-disposed practice-exercise element 285_{D} of the distal practice surface 281_{P} with the articulated arm 102 not in a retroflex position.

In some embodiments, as shown in the flowchart of FIG. 17B, the method can additionally comprise, before Step S23:

Step S25 orient the proximal exercise-element carrier 280_{P} so that the distally-facing practice surface 281_{P} is set to an angle between 45° and 135° relative to the platform plane 1100.

In some embodiments, as shown in the flowchart of FIG. 17C, the method can additionally comprise, before Step S24:

Step S25 orient the distal exercise-element carrier 280_{D} so that the distally-facing practice surface 281_{P} is set to an angle between 45° and 135° relative to the platform plane 1100.

In some embodiments, as shown in the flowchart of FIG. 17D, the method can additionally comprise, after Step S22 and before Step S23:

Step S26 retroflexing the articulated arm 102.

In embodiments, not all steps of any method need be carried out. In embodiments, steps of different methods can be beneficially combined.

It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art, which would occur to persons skilled in the art upon reading the foregoing description.

1. An assembly for performing practice exercises with a surgical device that comprises one or more articulated arms, the assembly comprising:
a. a support platform defining a platform plane;
b. an exercise-element carrier raised above the platform plane and comprising a first practice surface, the first practice surface comprising an array of one or more practice-exercise elements selected from the group consisting of holes, loops, pegs and hooks; and
c. an aperture raised above the platform plane and framed at least along an upper edge of the aperture, the aperture shaped for passage therethrough of a distal portion of an articulated arm,
wherein the exercise-element carrier and the aperture are arranged so that a practice-exercise element of the first practice surface is accessible to a distal end effector of the articulated arm when the articulated arm is in a retroflex position.

2. The assembly of claim 1, wherein the practice-exercise element of the first practice surface is accessible by the distal end effector of the articulated arm only when the articulated arm is in in the retroflex position.

3. The assembly of any preceding claim, wherein the first practice surface is oriented at an angle between 45° and 135° relative to the platform plane.

4. The assembly of any preceding claim, wherein the retroflex position is such that the articulated arm is flexed by at least 120° from an unflexed orientation.

5. The assembly of any preceding claim, wherein the retroflex position is such that the articulated arm is flexed by at least 150° from an unflexed orientation.

6. The assembly of any preceding claim, wherein the retroflex position is such that the articulated arm is flexed by at least 180° from an unflexed orientation.

7. The assembly of any preceding claim, wherein the aperture is formed through, under, or atop, the exercise-element carrier.

8. The assembly of claim 7, wherein the upper edge of the aperture is below the array of one or more practice-exercise elements of the first practice surface.

9. The assembly of claim 7, wherein a framed lower edge of the aperture is above the array of one or more practice-exercise elements of the first practice surface.

10. The assembly of any preceding claim, wherein the exercise-element carrier additionally comprises a second practice surface, the second practice surface comprising an array of one or more practice-exercise elements selected from the group consisting of holes, loops, pegs and hooks, wherein the array of one or more practice-exercise elements of the second practice surface is accessible to the distal end effector of the articulated arm when the articulated arm is not in a retroflex position.

11. The assembly of claim 10, wherein the first and second practice surfaces are parallel.

12. The assembly of claim 10, wherein the first and second practice surfaces are oriented at an acute angle to each other.

13. The assembly of any one of claims 10 to 12, wherein the second practice surface is oriented at an angle between 45° and 135° relative to the platform plane.

14. The assembly of any one of claims 10 to 13, wherein a practice-exercise element of the second practice surface is accessible to the distal end effector of the articulated arm when the articulated arm is in an S-shaped position.

15. A method of performing practice exercises with the surgical device, the method comprising:
a. providing an assembly according to any one of claims 1 to 9;
b. causing the distal portion of the articulated arm to traverse the aperture; and
c. with the articulated arm in the retroflex position, causing the distal end effector of the articulated arm to perform a practice exercise with at least one practice-exercise element of the first practice surface.

16. The method of claim 15, additionally comprising, before the causing of the distal end effector of the articulated arm to perform the practice exercise:
orienting the exercise-element carrier so that the first practice surface is set to an angle between 45° and 135° relative to the platform plane.

17. The method of either one of claim 15 or 16, additionally comprising, after the causing of the distal portion of the articulated arm to traverse the aperture, and before the causing of the distal end effector of the articulated arm to perform the practice exercise:
retroflexing the articulated arm.

18. A method of performing practice exercises with a surgical device that comprises one or more articulated arms, the method comprising:
a. providing an assembly according to any one of claims 10 to 14;
b. at a first time:
i. causing the distal portion of the articulated arm to traverse the aperture, and
ii. with the articulated arm in the retroflex position, causing the distal end effector of the articulated arm to perform a practice exercise with at least one of the practice-exercise elements of the first practice surface; and
c. at a second time, with the articulated arm not in a retroflex position, causing the distal end effector of the articulated arm to perform a practice exercise with at least one of the practice-exercise elements of the second practice surface.

19. The method of claim 18, additionally comprising, before the causing of the distal end effector of the articulated arm to perform the practice exercise with the at least one of the practice-exercise elements of the second practice surface:
orienting the exercise-element carrier so that the second practice surface is set to an angle between 45° and 135° relative to the platform plane.

20. An assembly for performing practice exercises with a surgical device that comprises one or more articulated arms, the assembly comprising:
a. a support platform defining a platform plane;
b. a proximal exercise-element carrier raised above the platform plane and comprising a distally-facing practice surface, the proximal exercise-element carrier having formed therethrough or thereunder an aperture shaped for passage therethrough of a distal portion of an articulated arm; and
c. a distal exercise-element carrier raised above the platform plane and comprising a proximally-facing practice surface,
wherein the aperture and the proximal and distal exercise panels are arranged so that (i) a distally-disposed practice-exercise element of the proximal exercise panel, selected from the group consisting of holes, loops, pegs and hooks, is accessible to a distal end effector of the articulated arm when the articulated arm is in a retroflex position, and (ii) a proximally-disposed practice-exercise element of the distal exercise panel, selected from the group consisting of holes, loops, pegs and hooks, is accessible to the distal end effector of the articulated arm when the articulated arm is not in a retroflex position.

21. The assembly of claim 20, wherein the distally-disposed practice-exercise element of the proximal exercise-element carrier is accessible to the distal end effector of the articulated arm only when the articulated arm is in a retroflex position.

22. The assembly of either one of claim 20 or 21, wherein the respective practice surfaces of the proximal and distal exercise panels are oriented at respective angles between 45° and 135° relative to the platform plane.

23. The assembly of any one of claims 20 to 22, wherein the retroflex position is such that the articulated arm is flexed by at least 120° from an unflexed orientation.

24. The assembly of one of claims 20 to 23, wherein the retroflex position is such that the articulated arm is flexed by at least 150° from an unflexed orientation.

25. The assembly of one of claims 20 to 24, wherein the retroflex position is such that the articulated arm is flexed by at least 180° from an unflexed orientation.

26. The assembly of one of claims 20 to 25, wherein an upper edge of the aperture is below the at least one practice-exercise element of the distally-facing practice surface of the proximal exercise panel.

27. The assembly of one of claims 20 to 25, wherein a lower edge of the aperture is above the at least one practice-exercise element of the proximally-facing practice surface of the distal exercise panel.

28. The assembly of any one of claims 20 to 27, wherein the proximally-disposed practice-exercise element of the distal exercise-element carrier is accessible to the distal end effector of the articulated arm when the articulated arm is in an S-shaped position.

29. A method of performing practice exercises with a surgical device that comprises one or more articulated arms, the method comprising:
a. providing an assembly according to any one of claims 20 to 28;
b. at a first time:
i. causing the distal portion of the articulated arm to traverse the aperture, and
ii. with the articulated arm in the retroflex position, causing the distal end effector of the articulated arm to perform a practice exercise with a distally-disposed practice-exercise element of the proximal exercise panel; and
c. at a second time, with the articulated arm not in a retroflex position, causing the distal end effector of the articulated arm to perform a practice exercise with a proximally-disposed practice-exercise element of the distal exercise panel.

30. The method of claim 29, additionally comprising, before the causing of the distal end effector of the articulated arm to perform the practice exercise with the distally-disposed practice-exercise element of the proximal exercise panel:
orienting the proximal exercise-element carrier so that the distally-facing practice surface is set to an angle between 45° and 135° relative to the platform plane.

31. The method of either one of claim 29 or 30, additionally comprising, before the causing of the distal end effector of the articulated arm to perform the practice exercise with the proximally-disposed practice-exercise element of the distal exercise panel:
orienting the distal exercise-element carrier so that the proximally-facing practice surface is set to an angle between 45° and 135° relative to the platform plane.

32. The method of either one of claim 29 or 30, additionally comprising, at the first time, after the causing of the distal portion of the articulated arm to traverse the aperture and before the causing of the distal end effector of the articulated arm to perform the practice exercise with the distally-disposed practice-exercise element of the proximal exercise panel:
retroflexing the articulated arm.
